(19) United States
(12) Patent Application Publication
(10) Pub. No.: US 2007/0293859 A1
(43) Pub. Date: Dec. 20, 2007

(54) Reconstruction device and method

(76) Inventor: Jeffrey C. Posnick, Potomac, MD (US)

Correspondence Address: STEPTOE & JOHNSON LLP, 1330 CONNECTICUT AVENUE, N.W., WASHINGTON, DC, 20036, US

(21) Appl. No.: 11/760,404

(22) Filed: Jun. 8, 2007

Related U.S. Application Data
(60) Provisional application No. 60/805,006, filed on Jun. 16, 2006.

(51) Int. Cl. A61B 17/00; A61F 5/04

(52) U.S. Cl. 606/57

(57) ABSTRACT

A device for mandible distraction includes a first temporal component having a temporal end, a second temporal component having a second temporal end, and a central anterior component in between the two and in front of the mandible. The device has locations on it to place distraction and stabilization pins that then connect to the mandible (on either side of an osteotomy through the mandible) for the purpose of distraction.

### CLAIM FOR PRIORITY

This application claims priority under 35 USC § 119(e) to U.S. Patent Application Ser. No. 60/805,006, filed on Jun. 16, 2006, which is incorporated by reference in its entirety.

### TECHNICAL FIELD

The present invention relates to mandibular reconstruction.

### BACKGROUND

Distraction is an appropriate method of treatment for certain medical conditions affecting the cranio-maxillofacial structures in general and the mandible in particular. The conditions of the mandible include but are not limited to Pierre Robyn sequence and Treacher Collins syndrome. Distraction is an appropriate method of treatment for medical conditions that involve the lower jaw (the mandible) which results in micrognathia. In subjects with Pierre Robyn sequence, the tongue tends to be displaced back towards the throat, where it can obstruct the airway (glossoptosis). Subjects with Pierre Robyn sequence, and many with Treacher Collins syndrome will also have a cleft palate.

### SUMMARY

In general, a mandibular distraction device can include a first temporal bar, a second temporal bar, at least one adjusting element and a central region between the first temporal bar and the second temporal bar. The central region can include a central anterior bar and two or more fixation elements for securing the distractor to a region of the mandible and for distracting the mandible toward the distraction device. A central anterior bar can be positioned transverse relative to the temporal bars. A temporal bar can include a substantially flat posterior temporal component and a substantially tubular lateral portion. A temporal bar can extend from a posterior temporal end attached to a side of a skull and then anteriorly in front of the mandible to an anterior end attached to the central anterior bar. The temporal bar can include a curve between a lateral portion and the central region. The second temporal bar can be adjustably positioned relative to the first temporal bar by altering a dimension, such as a length dimension of the central region.

In another aspect, a distraction device can include fixation elements such as fixation plates, screws, stabilization pins or distraction pins which may secure to the mandible through fixation plates and screws. A fixation element can be positioned on a temporal bar or the central bar. A fixation element can include a sliding attachment to the bar, a threaded hole, or a rod. Additional fixation elements can be secured at different locations on a bars for distracting the mandible after an osteotomy has been completed. The locations of the fixation elements can be unique, for example, locations can be customized for each subject based on the desired surgical plan.

In another aspect, a distraction device can include a temporal bar which can include a curve or angle between a posterior temporal component and a lateral portion. The first temporal bar and second temporal bar can be configured to curve around in front of the facial skeleton at the level of a mandible when the first temporal end is attached to one side of the skull and the second temporal end is attached to the other side of the skull. The curve can include an adjusting element or can be positioned between adjusting elements. The first temporal bar, second temporal bar, and central region can form a half-U-shape from a top perspective. The central region can include a central anterior bar, which can be configured to provide adjustment flexibility. A connecting joint of the first or second temporal bar (or both) and central region can be configured to provide adjustment flexibility. The temporal bar can be configured to receive a fixation element, such as a rod or a plurality of rods. The fixation elements can be configured to penetrate through a mandible at varied locations. The locations can be unique, for example, locations can be customized for each subject based on a desired surgical plan. The first temporal bar and the second temporal bar can be configured to be attached to the skull with at least one screw and at least one threaded hole. A temporal bar can be configured to receive a plurality of rods. A temporal bar can be configured to be attached to the skull with at least one screw and at least one threaded hole. The central anterior bar can include a variable height attachment post.

The temporal bar can be configured to receive a plurality of positions for securing the central anterior bar. A threaded hole or attachment can be located at each of the plurality of positions along the central anterior bar and then to the mandible.

A temporal bar can be configured to be adjustably paired with a second temporal bar using a rod, for example. The second temporal bar can be configured to be attached to the side of the skull opposite the side to which the temporal bar is attached. A first or second temporal bar (or both) can have a curved posterior end that can be configured to receive at least one fixation element.

A method for distracting a region of the facial skeleton can include attaching a curved temporal bar of a device to a skull such that a posterior temporal end of a temporal bar is attached to the skull at the temple-level and an anterior end of a temporal bar that curves around in front of the facial skeleton at the level of a mandible. The anterior end of the temporal bar can be secured to the mandible by applying a force to the mandible sufficient to effect distraction.

In other circumstances, a method for distracting a region of a facial skeleton, can include attaching a first temporal bar of a distractor to the skull, attaching a second temporal bar of a distractor to the skull, and securing a central anterior bar in a central region of the distractor to a mandible thereby applying a force to the mandible sufficient to effect distraction, wherein the second temporal bar is adjustably positioned relative to the first temporal bar by altering a dimension of the central region.

Advantageously, the distraction device can be easy to apply and remove, can be more comfortable for the subject, can have fewer moving parts, can be more secure to the skull base, and can provide protection to the mandible when compared to other devices used for mandibular distraction. It also can allow for oral food and liquid to be introduced with limited obstruction. Other devices can require more extensive dissection which may result in greater growth restriction, result in greater injury to the developing teeth and greater facial soft tissue scarring. With placement of stabilization fixation elements into the proximal mandible minimal compression force will occur within the temporomandibular joint as the distal segment of the mandible is distracted toward the anterior bar. This device also allows for an intraoperative naso-tracheal intubation tube to be used without interfering with device placement.

The details of one or more embodiments are set forth in the accompanying drawing and the description below. Other features, objects, and advantages will be apparent from the description and drawings, and from the claims.

### DESCRIPTION OF DRAWINGS

FIG. 1 is a drawing depicting a mandibular distraction device functionally attached to a human skull.

FIG. 2 is a drawing depicting a mandibular distraction device functionally attached to a human skull.

FIG. 3 is a drawing depicting a side view of a mandibular distraction device.

FIG. 4 is a drawing depicting a mandibular distraction device.

### DETAILED DESCRIPTION

In general, a method and apparatus for mandibular distraction includes attaching bars to the skull just above each external ear. Each bar can extend anteriorly along the outer surface of mandible, for example, the ramus, angle, body and symphysis. One or more osteotomies can be surgically completed through the mandible. Stabilization pins and distraction pins can then be secured at different locations on the bar. The pins perforate through the skin and then into the mandible. The locations in which they attach to the bar can be unique; for example, locations can be customized for each subject based on a desired surgical plan, taking into account individual features such as facial measurements and degree of skeletal deficiency, deformity, and/or asymmetry. The distraction or fixation pins can perforate through the skin of the face and then into the mandible. The pins can be secured into the mandible and can be used either to stabilize aspects of the mandible or distract portions of the mandible. Screws can stabilize the overall distraction device to the cranio-maxillofacial skeleton by inserting through the posterior aspect of each S-shaped temporal bar and then compressing against the skull. The screws can be positioned just above each external ear.

A mandibular distraction device can include a first temporal bar, a second temporal bar, at least one adjusting element and a central region between the first temporal bar and the second temporal bar, the central region including two or more distraction pins to be secured to the mandible. The temporal bar can include a substantially flat posterior temporal component and a substantially tubular lateral portion. The temporal bar can include a curve between a lateral portion and the central region. The second temporal bar can be adjustably positioned relative to the first temporal bar by altering a dimension of the central region.

From a side perspective, a first and second temporal bar can each have an S-shape. From a top perspective, each temporal bar can have a half-U shape. The S-shaped temporal bars can be attached to the subject's skull just above the external ears. Each S-shaped temporal bar can extend inferiorly and anteriorly adjacent to the outer surface of the mandible and then in front of the symphysis of the mandible in a location desirable for distraction of osteotomized components of the mandible.

Each S-shaped temporal bar can be designed to join to each other anteriorly through the central anterior bar. Stabilization pins and distraction pins can be secured to the bars and then perforate through the skin of the face into the mandible at locations that are customized for each subject. The locations can be unique, for example, locations can be customized for each subject based on a desired surgical plan. The pins can be positioned on either side of the osteotomized mandible in a manner appropriate to effect either stabilization or distraction of the mandible, according to a customized surgical plan.

The distraction device can include a metal or metal alloy, for example, titanium, aluminum, carbon, or other metal or metal alloy. Each component of the distraction device, independently, can be composed of titanium, aluminum, carbon, or other metal or metal alloy. However, it will be appreciated by those skilled in the art that other materials having suitable performance and bio-compatibility characteristics may be employed.

A distraction device can include fixation elements such as fixation plates, screws, stabilization pins or distraction pins, or a clamp at least partially configured to receive a pin or rod. A fixation element can be positioned on a temporal bar. A fixation element can include a sliding attachment to the bar, for example. Additional fixation elements can be secured at different locations on a bars for the purpose of effecting distraction of a mandible after an osteotomy has been completed.

Fixation plates can be secured with screws, directly or indirectly into the mandible. The fixation plates can include threaded fixtures for securing the stabilization pins and distraction pins that can pierce through the skin and secure to the S-shaped temporal bar and to the central anterior bar. Heavy wires can also be used to connect to the fixation plates and screws secured to the mandible and then to the S-shaped temporal bars or the central anterior bar.

A distraction device can include a temporal bar which includes a curve between a posterior temporal component and a lateral portion. The first temporal bar and second temporal bar can be configured to curve around in front of the facial skeleton at the level of the mandible when the first temporal bar has a first posterior temporal end attached to the skull and the second temporal bar has a second posterior temporal end attached to the skull. The central region can include an central anterior bar, configured to provide adjustment flexibility. The temporal bar can be configured to receive a rod or other fixation elements. The first temporal bar and the second temporal bar are configured to be attached to the skull with at least one screw and at least one threaded hole.

Each S-shaped temporal bar can be attached rigidly to one side of the subject's skull. Each S-shaped temporal bar can extend around the subject's mandible and attach to a central anterior bar in front of the symphysis of the mandible. The location of the bars can be positioned or configured as desired according to a unique plan for each subject to effect distraction. The two S-shaped temporal bars can be connected in the front of the mandible by a central anterior bar composed of one or more rods. The connecting joint of the S-shaped temporal bars and central anterior bar can provide adjustment flexibility. The rods of the central anterior bar can attach to the S-shaped temporal bars and provide a stable U-shaped overall distraction device which can be secured to the skull just above each external ear by screws.

Devices for internal mandible distraction are described, for example, in U.S. Pat. No. 6,113,599, which is incorporated by reference herein. Devices for mandible osteosynthesis are described, for example, in U.S. Pat. No. 6,423,068, which is incorporated by reference herein.

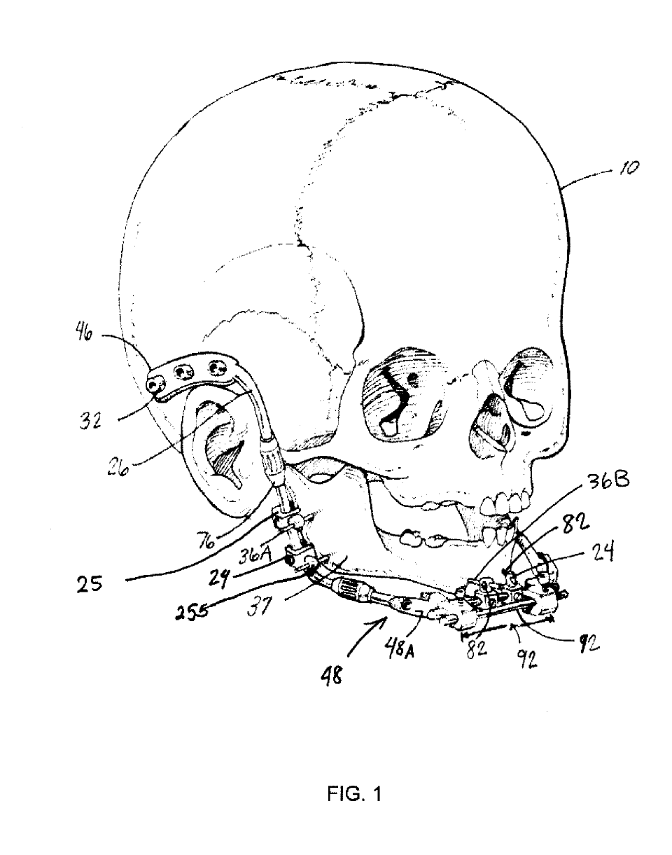

Referring to FIG. 1, a mandibular distraction device or distractor can be functionally attached to a human skull. The posterior end (46) of each S-shaped temporal bar (26) can be attached on one side of the human skull (10) at a point above the external ear. The S-shaped temporal bar can be attached to the human skull with screws (32), which can thread through holes or openings in the bar and then through a subjects scalp and compress against the base of the skull. The posterior end of the S-shaped temporal bar can exert sufficient pressure to achieve a compression force to the skull and can provide rigid stability to the temporal bar adjacent to the outside surface of the mandible. A compression force can stabilize each S-shaped temporal bar once connected by rods anteriorly in a central region (92). Stabilization pins (36A) and distraction pins (36B) can then be secured to the device. The stabilization pins and distraction pins may penetrate through the mandible at specific locations that are customized or unique for each subject. A stabilization pin and distraction pin can include a thread (25) or threaded hole (255) configured to receive a rod (36A, 36B). Each S-shaped temporal bar can extend anteriorly on the sides of the subject's mandible (37) and around the front of the face. Each S-shaped temporal bar can include at least one adjusting element (76) which can further change the length or tension of the bar. The S-shaped temporal bar can also include at least one connecting stabilization pin (24) or connecting distraction pin (82) which connect the bar to the stabilization or distraction pin.

A stabilization pin or distraction pin can be attached to the device and positioned at any location along the mandible. The stabilization pin or distraction pin can be clamped onto the bar and then into the mandible. The stabilization pin or distraction pin can be positioned on the central anterior bar in the central anterior region or on the S-shaped temporal bars of the distraction device. The stabilization pin or distraction pin can be positioned between the adjustable elements on the S-shaped temporal bar. The stabilization pin or distraction pin can be partially or completely wrapped or clamped around the S-shaped temporal bar or central anterior bar of the distraction device. The stabilization pin or distraction pin can include threads for attachment into the mandible according to a predetermined distraction plan for the specific subject. The pin can be for stabilizing the mandible (36A). The pin can clamp onto the bar (25) and then puncture the skin and attach to the subject's mandible. The pin can have a fixed or adjustable length. For example, a pin may be positioned orthogonal relative to the surface of the mandible. The pin can also be positioned at any angle relative to the surface of the mandible, depending on the force or direction of distraction uniquely desired for the subject. The pins can be positioned vertically or horizontally. Two or more pins can be aligned such that they are parallel to each other. Two or more pins can be positioned such that they form an angle. A distraction pin can be attached to the mandible in some other way. For example, the distraction pins can have a sliding attachment (24) on the bar. The mandibular distraction pin can be fixed or adjustable. The mandibular distraction pin can be configured to attach directly to the mandible or directly to a fixation plate or fixation screw which is attached to the mandible.

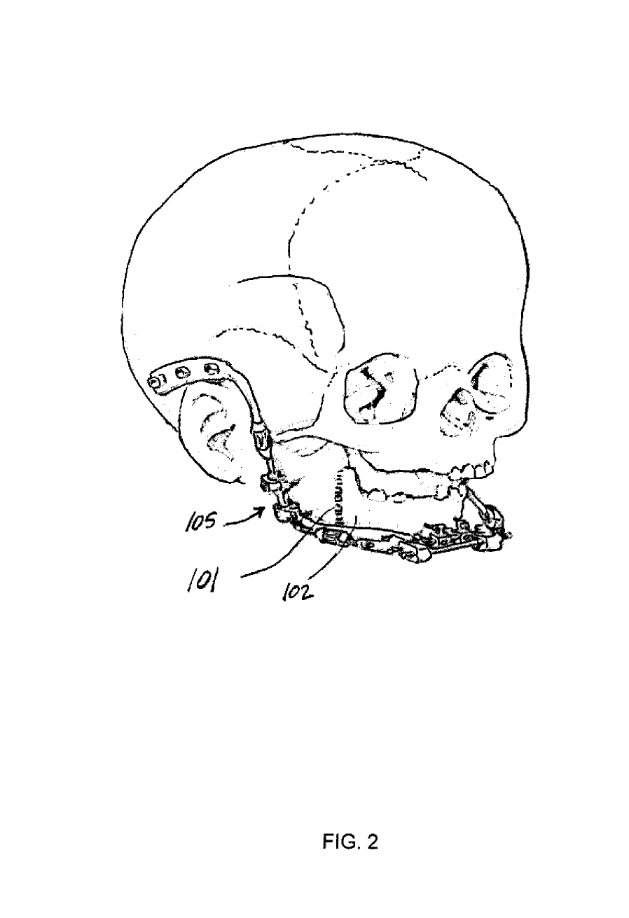

Referring to FIG. 2, a mandibular distraction device (105) can be used for distraction osteogenesis. In distraction osteogenesis, bone can be generated in the distraction gap (101), as a result of a tension-stress effect by the distraction device on the mandible. A tension-stress effect on the mandible across the osteotomy site can result in an increase in metabolic activity, an increase in cellular proliferation and a neovascular in-growth similar to normal endochondral ossification. Distraction osteogenesis can be performed in three dimensions: horizontal; transverse; and/or transverse vertical to achieve change in the basal-mandibular bone (102). For example, the direction of distraction, including the magnitude of movement can be tailored or customized to each subject. The calculated force and distance of distraction can determine specific placement of fixation elements, adjustment elements, applied stress and compression forces for the subject, taking into account size differentials, shape variations and possible facial asymmetry.

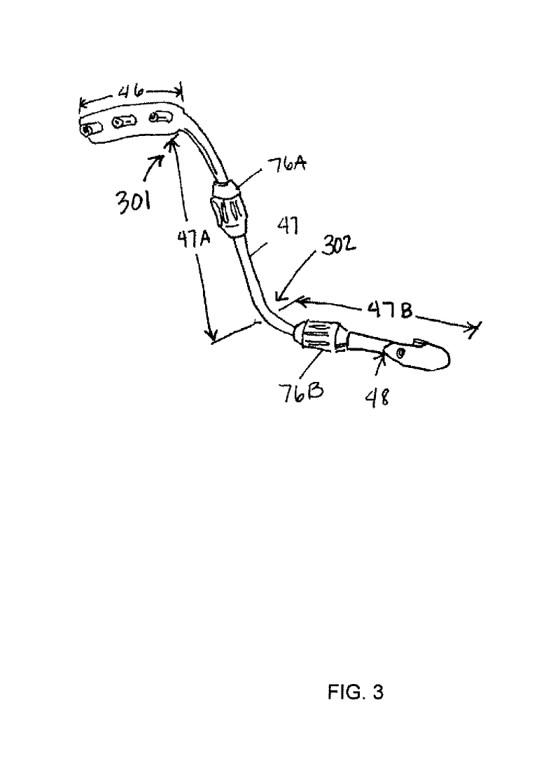

Referring to FIG. 3 from a side view, the mandibular distraction device can have an S-shape. The S-shape can include at least a first curve (301) and a second curve (302). The first curve can form an arch or angle. On either side of the arch or angle, there can be a posterior temporal component (46) and a superiorly lateral portion (47A) of the S-shaped temporal bar. The first curve can include a superior adjusting element (76A). Alternatively, a first curve can be positioned between a posterior temporal component (46) and a superior adjusting element (76A).

A second curve (302) can form an arch or an angle formed by a superior-lateral portion (47A) and an inferior lateral portion (47B) of the S-shaped temporal bar.

An adjustable angle (48) can be formed between an inferior lateral component of the S-shaped temporal bar and the central anterior region. The central anterior bar can include at least one metal rod.

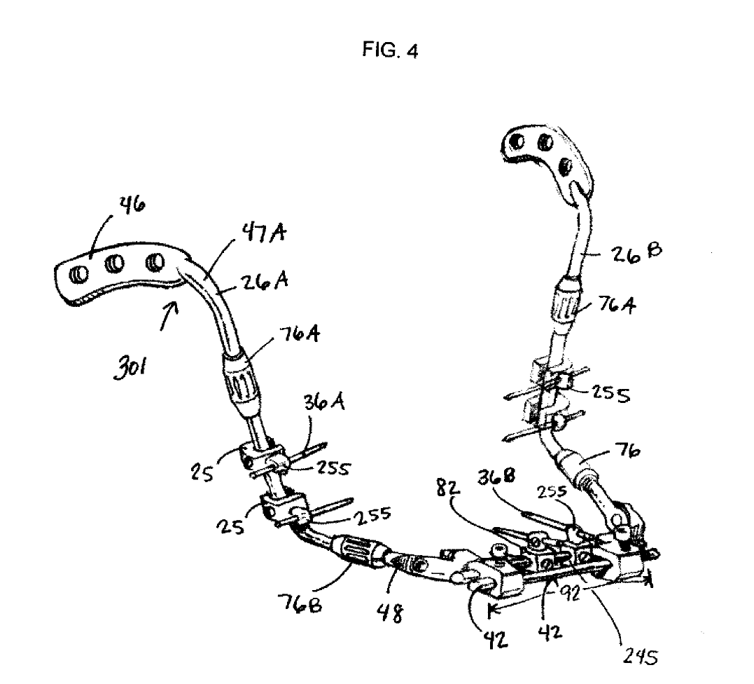

Referring to FIG. 4, a first temporal bar (26A) and second temporal bar (26B) may be positioned and repositioned according to a distraction plan, customized for the surgical plan unique for the subject. By placing stabilization pins (36A), which attach to the mandible on one side of the osteotomy and distraction pins (36B) attached on the other side of the osteotomy a force is applied to the mandible which allows distraction towards the central anterior bar (92). A distraction pin can be attached to the central anterior bar (92, 42) in various ways that are unique for the subject. For example, the distraction pins can have a sliding attachment (245) on the bar. The distraction pins can also contact or attach to fixation plates securing to the mandible. The mandibular distraction pin can be fixed or adjustable. The mandibular distraction pin can be configured to attach directly to the mandible or directly to a fixation plate or fixation screw which is attached to the mandible. Two or more S-shaped temporal bars (26A, 26B) can meet anteriorly and join at the central anterior region (92), including a central anterior bar (42). The S-shaped temporal bar can be joined anteriorly to the central anterior bar, which can include one or more threaded or non-threaded rods (36A, 36B), which has an additional adjustable angle (48) to further control the mandibular distraction process.

Other embodiments are within the scope of the following claims.

1. A mandibular distraction device comprising:
a first temporal bar,
a second temporal bar;
at least one adjusting element; and
a central region between the first temporal bar and the second temporal bar, the central region including two or more fixation elements for distracting a mandible to the device, the temporal bars including two or more fixation elements for stabilizing the mandible to the device.

2. The device of claim 1, wherein the first temporal bar includes a substantially flat posterior temporal component and a substantially tubular lateral portion.

3. The device of claim 1, wherein the first temporal bar includes a curve between a lateral portion and the central region.

4. The device of claim 1, wherein the second temporal bar includes a substantially flat posterior temporal component and a substantially tubular lateral portion.

5. The device of claim 1, wherein the second temporal bar includes a curve between a lateral portion and the central region.

6. The device of claim 1, wherein the first and second temporal bar includes a substantially flat posterior temporal component and a substantially tubular lateral portion.

7. The device of claim 1, wherein the first and second temporal bar includes a curve between a lateral portion and the central region.

8. The device of claim 1, wherein the second temporal bar is adjustably positioned relative to the first temporal bar by altering a dimension of the central region.

9. The device of claim 1, wherein the fixation elements include fixation plates, screws, stabilization pins or distraction pins.

10. The device of claim 9, further comprising at least one fixation element on a temporal bar.

11. The device of claim 1, wherein the temporal bar includes a curve between a posterior temporal component and a lateral portion.

12. The device of claim 1, wherein the first temporal bar and second temporal bar are configured to curve around in front of a facial skeleton at the level of the mandible when the first temporal bar has a first posterior temporal end attached to a skull and the second temporal bar has a second posterior temporal end attached to the skull.

13. The device of claim 1, wherein the central region includes a central anterior bar.

14. The device of claim 13, wherein the central anterior bar is configured to provide adjustment flexibility.

15. The device of claim 1, wherein the fixation elements are configured to penetrate through a mandible at customized locations that are specific for a subject.

16. The device of claim 1, wherein a connecting joint of the first or second temporal bar and the central region is configured to provide adjustment flexibility.

17. The device of claim 1, wherein the first or second temporal bar is configured to receive a rod.

18. The device of claim 1, wherein the first or second temporal bar is configured to receive a plurality of rods.

19. The device of claim 1, wherein the first temporal bar and the second temporal bar are configured to be attached to a skull with at least one screw and at least one threaded hole.

20. The device of claim 3, wherein the curve includes an adjusting element or is positioned between adjusting elements.

21. The device of claim 1, wherein the first temporal bar, second temporal bar, and central region form a half-U-shape from a top perspective.

22. The device of claim 10, wherein the fixation elements include a sliding attachment on the bar.

23. The device of claim 1, further comprising additional fixation elements secured at different locations on the bars for distracting and stabilizing the mandible after an osteotomy has been completed.

24. The device of claim 19, wherein the each bar has a curved posterior end configured to receive at least one fixation element.

25. A method for distracting a region of a mandible comprising:
attaching a curved temporal bar of a device to a skull such that a posterior temporal end of a temporal bar is attached to the skull at substantially temple-level above an external ear and an anterior end of a distraction device curves around in front of the mandible; and
securing an anterior end of the distraction device to the mandible thereby applying a force to an attached region sufficient to effect distraction.

26. The method of claim 25, wherein securing the anterior end of the device to a mandible includes attachment with an adjustable distraction screw, rod, or pin.

27. The method of claim 26, wherein the distraction screw, rod, or pin is adjustably secured to the mandible and to the central anterior bar component of the device.

28. The method of claim 25, wherein the temporal bar is configured to provide a plurality of positions for securing the central anterior bar of the device to the mandible.

29. The method of claim 28, wherein a threaded hole or attachment is located at each of the plurality of positions along the central anterior bar and then to the mandible.

30. The method of claim 25, wherein the temporal bar is configured to receive a stabilization rod, fixation rod, or screw.

31. The method of claim 30, wherein the central anterior bar includes a variable height attachment post.

32. The method of claim 25, wherein the temporal bar is configured to be attached to the skull with screws.

33. The method of claim 25, wherein the temporal bar extends from the posterior temporal end attached to the side of the skull and extends anterior to the front of the mandible.

34. The method of claim 33, wherein the temporal bar includes an adjustable anterior central component of the device.

35. The method of claim 25, wherein the temporal bar is configured to be adjustably paired with a second temporal bar, the second temporal bar configured to be attached to the side of the skull opposite the side to which the temporal bar is attached.

36. The method of claim 25, wherein the temporal bar is configured to be adjustably paired with a second temporal bar.

37. The method of claim 36, wherein the temporal bar is configured to be adjustably paired with a second temporal bar with at least a rod.

38. A method for distracting a region of a mandible comprising:
attaching a first temporal bar of a distraction device to a skull;
attaching a second temporal bar of a distraction device to the skull; and
securing a central anterior bar in a central region of the distraction device to a mandible, thereby applying a force to the mandible sufficient to effect distraction,
wherein the second temporal bar is adjustably positioned relative to the first temporal bar by altering a dimension of the central region.

39. The method of claim 38, wherein the device further comprises a first curved posterior end and a second curved posterior end.

40. The method of claim 39, wherein the first temporal bar and second temporal bar are curved.

41. The method of claim 39, wherein the first temporal bar and second temporal bar are configured to curve around in front of the facial skeleton at the level of a mandible when the first posterior temporal end is attached to the skull and the second posterior temporal end is attached to the skull.

42. The method of claim 38, wherein securing the central anterior bar of the device to a mandible includes attaching a distraction screw, pin or rod adjustably secured to the central anterior bar of the device.

43. The method of claim 42, wherein the distraction screw, pin, or rod is adjustably secured to the rod of the central anterior bar of the device.

44. The method of claim 38, wherein the central anterior bar of the device includes a plurality of positions for securing the device to the mandible of the facial skeleton.

45. The method of claim 44, wherein a threaded hole or attachment is located at each of the plurality of positions on the central anterior bar of the device.

46. The method of claim 39, wherein the temporal bar is configured to receive a stabilization pin or distraction pin.

47. The method of claim 46, wherein the central anterior bar includes a variable height attachment post.

48. The method of claim 38, wherein the first temporal bar and the second temporal bar are configured to be attached to the skull with screws.

49. The method of claim 39, wherein the first temporal bar extends from a first posterior temporal end attached to a first side of the skull and then anteriorly in front of the mandible attached to the central anterior bar and the second temporal bar extends from the second posterior temporal end attached to a second side of the skull and to the central anterior bar in front of the mandible.

50. The method of claim 49, wherein the first temporal bar and second temporal bar include an adjustable central anterior bar.

51. The method of claim 39, wherein the first temporal bar and second temporal bar are configured to be adjustably paired.

52. The method of claim 51, wherein the first temporal bar and second temporal bar are configured to be adjustably paired with at least a central rod which connects the first and second temporal bars together.
